(19) United States
(12) Patent Application Publication
(10) Pub. No.: US 2009/0264905 A1
(43) Pub. Date: Oct. 22, 2009

(54) Medical Instrument

(76) Inventor: Masaki Funada, Mie (JP)

Correspondence Address: Kubotera & Associates, 200 Daingerfield Road, Suite 202, Alexandria, VA, 22314, US

(21) Appl. No.: 12/227,068

(22) PCT Filed: May 24, 2007
(86) PCT No.: PCT/JP2007/060625

§ 371 Date: Nov. 6, 2008

(30) Foreign Application Priority Data
May 30, 2006 (JP) 2006-149069

(51) Int. Cl. A61B 17/06

(52) U.S. Cl. 606/146; 606/144

(57) ABSTRACT

A medical instrument which allows ligation in a living body to be efficiently carried out in safety by only one operator, or one operator and an assistant is provided. The medical instrument comprises a puncture needle for surgical suture insertion (20); a puncture needle for surgical suture gripping (30); a stylet (40) which is slidably inserted into the inside of the puncture needle for surgical suture gripping (30); and a fixing member (50) to which the puncture needle for surgical suture insertion (20) and the puncture needle for surgical suture gripping (30) are fixed, and when an annular member (42) which is formed at the distal end of the stylet (40) is projected from the distal end of the puncture needle for surgical suture gripping (30), the annular member (42) is extended such that the central axis of the puncture needle for surgical suture insertion (20) or its extension line penetrates through the inside of the annular member (42), with a feeding-out mechanism (60) which sequentially feeds out a surgical suture (S) toward the distal end thereof being provided on the proximal end side of the puncture needle for surgical suture insertion (20), and a projection mechanism (70) which causes the annular member (42) accommodated in the inside of the puncture needle for surgical suture gripping (30) to be projected from the distal end of the puncture needle for surgical suture gripping (30) being provided on the proximal end side of the puncture needle for surgical suture gripping (30).

### TECHNICAL FIELD

The present invention relates to a medical instrument which is used when a catheter is transcutaneously inserted into an abdomen viscus. Particularly, the present invention relates to a medical instrument which is used to fix the front abdominal wall and the splanchnic wall to each other for facilitating insertion of a catheter in endoscopic gastrostomy which is carried out for such a purpose as nutrient supply, body fluid draining, or the like.

### BACKGROUND ART

Conventionally, for patients who need long-term administration of nutritions, laparotomic gastrostomy has been carried out. However, a general gastrostomy technique involves excessive surgical invasiveness, thus, depending upon the condition of the patient, it is often impossible to make operation. Then, recently, gastrostomy techniques which minimize the surgical invasiveness have been devised, and a variety of medical instruments for accomplishing them have been proposed.

For example, the present inventor has already proposed a medical instrument, comprising a puncture needle for surgical suture insertion; a puncture needle for surgical suture gripping that is provided substantially in parallel with the puncture needle for surgical suture insertion, being separated therefrom a prescribed distance; a stylet which is slidably inserted into the inside of the puncture needle for surgical suture gripping; and a fixing member to which the basal end portions of the puncture needle for surgical suture insertion and the puncture needle for surgical suture gripping are fixed (for example, referring to Japanese Examined Patent Application Publication No. H06-024533 (JP 6-24533 B2 (1994))).

### DISCLOSURE OF THE INVENTION

However, even with the aforementioned prior art invention, for carrying out a general gastrostomy, there has been a need for one operator, one endoscope operator, and one assistant for manipulating the medical instrument, and in a hospital facility where no endoscope operator is available, it has been difficult to carry out gastrostomy. In addition, the personal expenses required have been high. Therefore, about the medical instrument, there has been a demand for further simplifying the operation to improve the convenience, thereby to allow gastrostomy to be performed with ease even in a hospital facility where no endoscope operator is available, and reduce the personal expenses which would be required.

The present invention has been made in view of such problems which have been faced by the prior art, and is intended to provide a medical instrument with which the operation of sequentially feeding out the surgical suture inserted into the inside of the puncture needle for surgical suture insertion toward the distal end thereof, and the operation of projecting the annular member of the stylet that is accommodated in the inside of the puncture needle for surgical suture gripping, from the distal end thereof, or withdrawing the projected annular member can be easily carried out even by one hand, whereby only one operator is capable of performing ligation and endoscope manipulation, which allows only one operator or one operator and an assistant to efficiently carry out the operation in safety.

The subject matters of the present invention to achieve the above purpose are disclosed in the following respective aspects of the present invention:

According to a first aspect of the present invention, a medical instrument, comprising a puncture needle for surgical suture insertion; a puncture needle for surgical suture gripping that is provided substantially in parallel with the puncture needle for surgical suture insertion, being separated therefrom a prescribed distance; a stylet which is slidably inserted into the inside of the puncture needle for surgical suture gripping; and a fixing member to which the puncture needle for surgical suture insertion and the puncture needle for surgical suture gripping are fixed, wherein

the stylet is provided with an annular member at the distal end thereof that is made of an elastic material, and is accommodatable in the inside of the puncture needle for surgical suture gripping, and when the annular member is projected from the distal end of the puncture needle for surgical suture gripping, the annular member is extended toward the puncture needle for surgical suture insertion such that the central axis of the puncture needle for surgical suture insertion or its extension line penetrates through the inside of the annular member;

on the proximal end side of the puncture needle for surgical suture insertion, a feeding-out mechanism for sequentially feeding out a surgical suture inserted into the inside of the puncture needle for surgical suture insertion from the proximal end thereof toward the distal end thereof is provided; and

on the proximal end side of the puncture needle for surgical suture gripping, a projection mechanism for projecting the annular member of the stylet, which is accommodated in the inside of the puncture needle for surgical suture gripping, from the distal end of the puncture needle for surgical suture gripping is provided.

According to a second aspect of the present invention, the medical instrument according to the first aspect, wherein the puncture needle for surgical suture insertion is fixed to the fixing member with the proximal end thereof being open to the fixing member;

the feeding-out mechanism includes a feeding-out guide member which is fixed to the fixing member in a location where the proximal end of the puncture needle for surgical suture insertion is open to the fixing member; and

the feeding-out guide member is provided with a guide bore which is communicated with the proximal end of the puncture needle for surgical suture insertion, and a roller which is turnably disposed, being butted against on the way of the surgical suture that is guided into the inside of the puncture needle for surgical suture insertion through the guide bore, and which sequentially feeds out the surgical suture toward the distal end of the puncture needle for surgical suture insertion through the turning operation thereof.

According to a third aspect of the present invention, the medical instrument according to the first aspect, wherein the puncture needle for surgical suture gripping is fixed to the fixing member with the proximal end thereof being open to the fixing member;

the projection mechanism includes a projection guide member which is fixed to the fixing member in a location where the proximal end of the puncture needle for surgical suture gripping is open to the fixing member; and

the projection guide member is cylindrically formed so as to movably guide a pushbutton in a linear direction that is disposed on the proximal end side of the stylet, being provided with urging means which urges the pushbutton toward the proximal end thereof to accommodate the annular member in the inside of the puncture needle for surgical suture gripping, and with locking means which allows the pushbutton, which is depressed toward the distal end thereof against the urging force of the urging means to project the annular member from the distal end of the puncture needle for surgical suture gripping, to be temporarily seized in a locking position.

According to a fourth aspect of the present invention, the medical instrument according to the first aspect, wherein the puncture needle for surgical suture insertion and the puncture needle for surgical suture gripping are provided with a flat plate-like member through which they are slidably penetrated; and

to the flat plate-like member, the distal end of an operating lever which extends substantially in parallel with the puncture needle for surgical suture insertion and the puncture needle for surgical suture gripping is fixed, the operating lever being slidably penetrated through the fixing member on the way, and a handle for sliding manipulation of the flat plate-like member being provided at the proximal end of the operating lever.

According to a fifth aspect of the present invention, the medical instrument according to the first aspect, wherein two sets of the puncture needle for surgical suture insertion and the feeding-out mechanism, and two sets of the puncture needle for surgical suture gripping and the projection mechanism are provided.

The present invention functions as follows:

According to the medical instrument pertaining to the present invention, in carrying out ligation of two biological membranes which are laid one upon the other with the distal end of the puncture needle for surgical suture insertion and that of the puncture needle for surgical suture gripping being punctured in a living body, the projection mechanism provided on the proximal end side of the puncture needle for surgical suture gripping causes the annular member provided at the distal end of the stylet accommodated in the inside of the puncture needle for surgical suture gripping to be projected from the distal end of the puncture needle for surgical suture gripping. Then, the annular member is extended toward the puncture needle for surgical suture insertion such that the central axis of the puncture needle for surgical suture insertion or its extension line penetrates through the inside of the annular member.

Then, the feeding-out mechanism provided on the proximal end side of the puncture needle for surgical suture insertion is used to sequentially feed out the surgical suture inserted from the proximal end of the puncture needle for surgical suture insertion into the inside thereof toward the distal end thereof to cause the surgical suture to be projected from the distal end of the puncture needle for surgical suture insertion and passed through the inside of the annular member. Thereafter, the stylet is withdrawn to accommodate the annular member in the inside of the puncture needle for surgical suture gripping. Through such a manipulation, the annular member is deformed such that the annular space thereof is gradually narrowed down, the surgical suture being held, and finally the portion of the surgical suture that is held by the annular member is accommodated in the inside of the puncture needle for surgical suture gripping together with the annular member.

In this way, the operation of feeding out the surgical suture and the operation of projecting or withdrawing the annular member from/into the distal end of the puncture needle for surgical suture gripping, which require fine and careful manipulation, can be reliably and easily performed by means of the feeding-out mechanism and the projection mechanism, respectively, thus in case of making gastrostomy, even only one operator is capable of performing stomach wall fixing and endoscope manipulation, which allows only one operator or one operator and an assistant to efficiently carry out gastrostomy in safety.

Specifically for example, if the feeding-out mechanism includes a feeding-out guide member which is fixed to the fixing member in a location where the proximal end of the puncture needle for surgical suture insertion is open to the fixing member, and this feeding-out guide member is provided with a guide bore which is communicated with the proximal end of the puncture needle for surgical suture insertion, and a roller which is turnably disposed, being butted against on the way of the surgical suture that is guided into the inside of the puncture needle for surgical suture insertion through the guide bore, and which, through the turning operation, sequentially feeds out the surgical suture toward the distal end of the puncture needle for surgical suture insertion, the surgical suture can be easily fed out by an exact desired amount by simply turning the roller by finger.

In addition, specifically for example, if the projection mechanism includes a projection guide member which is fixed to the fixing member in a location where the proximal end of the puncture needle for surgical suture gripping is open to the fixing member, and this projection guide member is cylindrically formed so as to movably guide a pushbutton in a linear direction that is disposed on the proximal end side of the stylet, being provided with urging means which urges the pushbutton toward the proximal end thereof to accommodate the annular member in the inside of the puncture needle for surgical suture gripping, and with locking means which allows the pushbutton, which is depressed toward the distal end thereof against the urging force of the urging means to project the annular member from the distal end of the puncture needle for surgical suture gripping, to be temporarily seized in a locking position, the annular member can be easily projected to an exact position by simply depressing the pushbutton by finger.

In addition, if the puncture needle for surgical suture insertion and the puncture needle for surgical suture gripping are provided with a flat plate-like member through which they slidably penetrated, and to the flat plate-like member, the distal end of an operating lever which extends substantially in parallel with the puncture needle for surgical suture insertion and the puncture needle for surgical suture gripping is fixed, the operating lever being slidably penetrated through the fixing member on the way, and a handle for sliding manipulation of the flat plate-like member being provided at the proximal end of the operating lever, it can be avoided that, at the time of puncture, the positional relationship between the puncture needle for surgical suture insertion and the puncture needle for surgical suture gripping is changed, and more specifically, that the distance between the respective puncture needles is changed (for example, shortened), and in addition, gripping the handle to slide it will move the flat plate-like member to a desired position, which can eliminate a possibility that, at the time of puncture, the flat plate-like member may obstruct the puncture operation.

Further, in case where two sets of the puncture needle for surgical suture insertion and the feeding-out mechanism, and two sets of the puncture needle for surgical suture gripping and the projection mechanism are provided, the abdominal wall and the gastric corpus front wall, for example, can be easily, rapidly, safely, and reliably fixed to each other in two places at the same time, thus the number of times of puncture invasiveness of this medical instrument into the patient required in this fixing can be only one, which minimizes the burden on the patient.

### BRIEF DESCRIPTION OF THE DRAWINGS

FIG. 1 is a sectional view illustrating a medical instrument pertaining to one embodiment of the present invention;

FIG. 2 is a sectional view illustrating the medical instrument pertaining to one embodiment of the present invention that is in the state in which the annular member of the distal end portion of the stylet is projected;

FIG. 3 is a side view illustrating the medical instrument pertaining to one embodiment of the present invention;

FIG. 4 is a top view illustrating the medical instrument pertaining to one embodiment of the present invention;

FIG. 5 is an explanatory drawing for explaining the function of the medical instrument pertaining to one embodiment of the present invention;

FIG. 6 is an explanatory drawing for explaining the function of the medical instrument pertaining to one embodiment of the present invention;

FIG. 7 is an explanatory drawing for explaining the function of the medical instrument pertaining to one embodiment of the present invention;

FIG. 8 is an explanatory drawing for explaining the function of the medical instrument pertaining to one embodiment of the present invention;

FIG. 9 is an explanatory drawing for explaining the function of the medical instrument pertaining to one embodiment of the present invention;

FIG. 10 is an explanatory drawing for explaining the function of the medical instrument pertaining to one embodiment of the present invention;

FIG. 11 is an explanatory drawing for explaining the function of the medical instrument pertaining to one embodiment of the present invention; and

FIG. 12 is a perspective view illustrating the medical instrument pertaining to one embodiment of the present invention that is loaded with a cover body.

### BEST MODE FOR CARRYING OUT THE INVENTION

Hereinbelow, one exemplary embodiment of the present invention will be described with reference to the drawings.

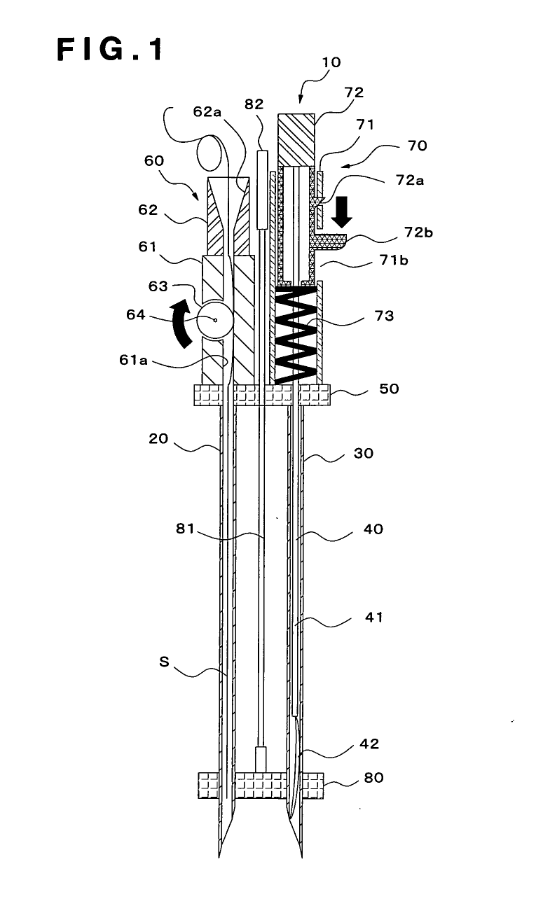

FIG. 1 to FIG. 12 show the one embodiment of the present invention.

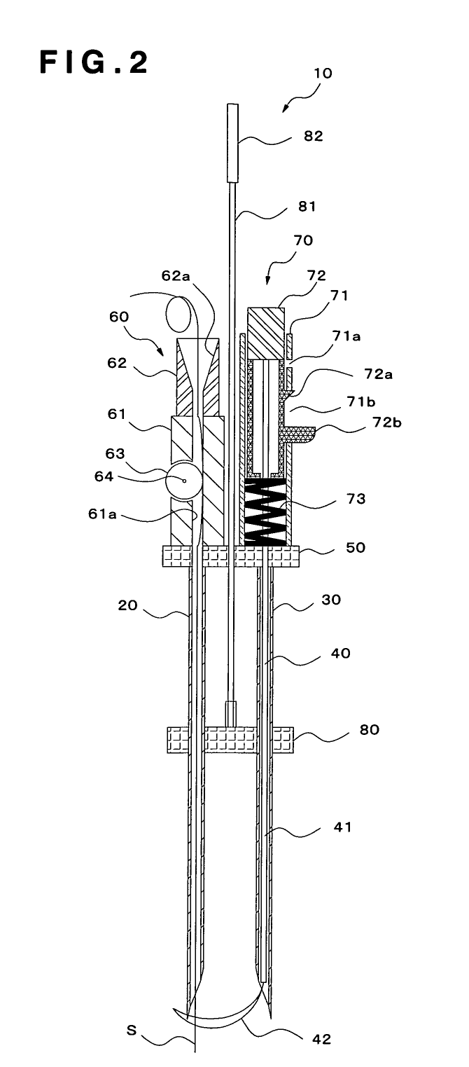

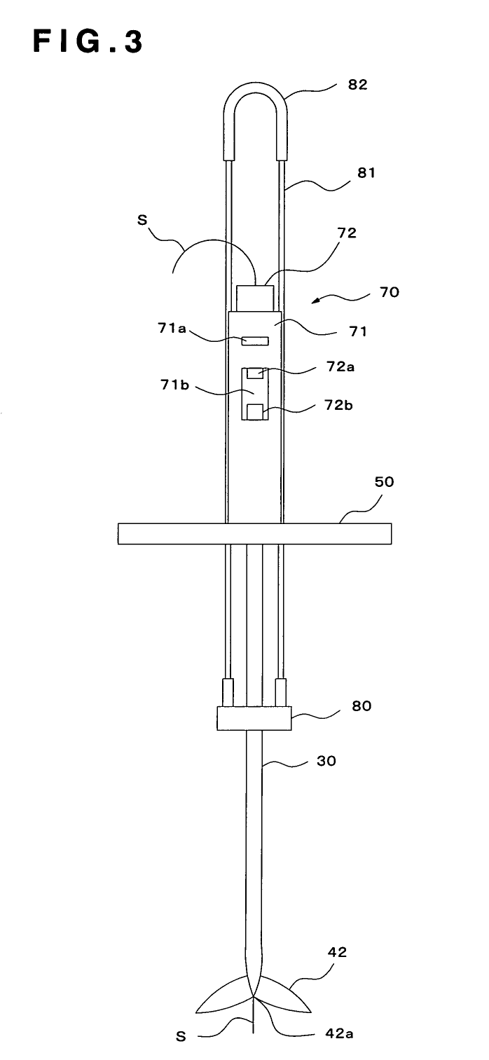

FIG. 1 and FIG. 2 are longitudinal sectional view of a medical instrument 10 pertaining to the present embodiment, and FIG. 3 is a side view of the medical instrument 10.

As shown in FIG. 1 and FIG. 2, the medical instrument 10 includes a puncture needle for surgical suture insertion 20; a puncture needle for surgical suture gripping 30 which is provided in substantially parallel with the puncture needle for surgical suture insertion 20, being separated therefrom a prescribed distance; a stylet 40 which is slidably inserted into the inside of the puncture needle for surgical suture gripping 30; and a fixing member 50 for fixing the basal end portions of the puncture needle for surgical suture insertion 20 and the puncture needle for surgical suture gripping 30. These will be sequentially described below.

The puncture needle for surgical suture insertion 20 is hollow to allow a surgical suture S to be inserted into the inside thereof, being made of a metal, such as stainless steel, and having a blade face for skin puncture at the distal end thereof. In the present embodiment, the distal end of the puncture needle for surgical suture insertion 20 has a blade face which is formed by cutting the puncture needle for surgical suture insertion 20 along a plane slantwise intersecting with the axis thereof. In addition, the inside of the puncture needle for surgical suture insertion 20 provides a passage for guiding a surgical suture S from the proximal end of the puncture needle for surgical suture insertion 20 to the distal end thereof in one direction.

The puncture needle for surgical suture insertion 20 is fixed to the fixing member 50 together with the puncture needle for surgical suture gripping 30 such that the proximal end of the puncture needle for surgical suture insertion 20 is open to the fixing member 50. The puncture needle for surgical suture insertion 20 may have any outside diameter and length, provided that it allows skin puncture and insertion of a surgical suture S, however, specifically, the outside diameter is preferably 21 G (gauge) to 17 G or so, and is more preferably 20 G to 18 G, and the length is preferably 70 mm to 120 mm or so, and is more preferably 80 mm to 100 mm.

On the proximal end side of the puncture needle for surgical suture insertion 20, a feeding-out mechanism 60 for sequentially feeding out the surgical suture S toward the distal end thereof that is inserted into the puncture needle for surgical suture insertion 20 from the proximal end thereof is provided. The feeding-out mechanism 60 includes a feeding-out guide member 61 which is fixed to the fixing member 50 in a location where the proximal end of the puncture needle for surgical suture insertion 20 is open to the fixing member 50. In the inside of the feeding-out guide member 61, a guide bore 61a which is communicated with the proximal end of the puncture needle for surgical suture insertion 20 is formed.

The guide bore 61a of the feeding-out guide member 61 extends along the axis of the puncture needle for surgical suture insertion 20, and to the proximal end of the feeding-out guide member 61, a hollow block 62 is mounted integrally therewith. An inside passage 62a of this block 62 is formed in a tapered shape, being gradually flared toward the basal end side thereof to allow the surgical suture S to be easily inserted. The feeding-out guide member 61 and the block 62 is made of a synthetic resin, such as vinyl chloride resin, a polyolefin, such as polypropylene or polyethylene, or polycarbonate.

The feeding-out guide member 61 is internally provided with a roller 63 which is turnably disposed, being butted against on the way of the surgical suture S to guide it into the inside of the puncture needle through the guide bore 61a for surgical suture insertion 20, and to sequentially feed it out toward the distal end of the puncture needle for surgical suture insertion 20 through the turning operation thereof. More specifically, the roller 63 is turnably pivoted in the inside of the feeding-out guide member 61 by the pivot 64, being capable of butting against the surgical suture S, sandwiching it between the face of the roller 63 on one side thereof and the internal wall surface in the inside of the guide bore 61a, while, on the other side thereof, being exposed to the circumferential surface of the feeding-out guide member 61 such that the roller 63 can be turned by finger.

The puncture needle for surgical suture gripping 30 is hollow to slidably insert the stylet 40 thereinto, being made of a metal, such as stainless steel, and having a blade face for skin puncture at the distal end thereof. The puncture needle for surgical suture gripping 30 used may be the same as the puncture needle for surgical suture insertion 20 aforementioned, or a puncture needle for surgical suture gripping having an equivalent outside diameter. Further, in order to allow the annular member 42 of a later described stylet 40 to reliably extend toward the puncture needle for surgical suture insertion 20, it is preferable that the blade face of the puncture needle for surgical suture gripping 30 at the distal end thereof be open toward the puncture needle for surgical suture insertion 20 as shown in FIG. 1.

The puncture needle for surgical suture gripping 30 is fixed to the fixing member 50 together with the puncture needle for surgical suture insertion 20 such that the proximal end of the puncture needle for surgical suture gripping 30 is open to the fixing member 50. The puncture needle for surgical suture gripping 30 may have any outside diameter and length, provided that it is capable of skin puncturing and allows insertion of the stylet 40, however, particularly, it is preferable that the puncture needle for surgical suture gripping 30 used have the same outside diameter and length as those of the puncture needle for surgical suture insertion 20. In addition, it is preferable that the distal end of the puncture needle for surgical suture gripping 30 that faces the puncture needle for surgical suture insertion 20 be chamfered to prevent the suture from being cut.

The stylet 40 is slidably inserted into the inside of the puncture needle for surgical suture gripping 30, being comprised of a rod-like member 41 having an outside diameter smaller than the inside diameter of the puncture needle for surgical suture gripping 30, and an annular member 42 which is fixed to the distal end of this rod-like member 41. To the basal end portion of the rod-like member 41, a pushbutton 72 of a later described projection mechanism 70 is fixed integrally therewith. The annular member 42 is made of an elastic material, and when projected from the distal end of the puncture needle for surgical suture gripping 30, the annular member 42 becomes annular, as shown in FIG. 2 and FIG. 3, while when not projected, it is deformed as shown in FIG. 1 to be substantially linear, being accommodated in the inside of the puncture needle for surgical suture gripping 30.

The rod-like member 41 and annular member 42 of the stylet 40 is slidable in the inside of the puncture needle for surgical suture gripping 30. When the annular member 42 of the stylet 40 is projected from the distal end of the puncture needle for surgical suture gripping 30, it is extended toward the puncture needle for surgical suture insertion 20 such that the central axis of the puncture needle for surgical suture insertion 20 or its extension line penetrates through the inside of the annular member 42 as shown in FIG. 2 and FIG. 3.

More specifically, as shown in FIG. 3, the annular member 42 is fixed to the distal end of the rod-like member 41 at a certain angle, and further it is preferable that the annular member 42 have a curving shape which, when viewed from the side, provides a bottom in the central portion or in a portion which is off the central portion, being slightly closer to the tip of the shape. By thus configuring the annular member 42, the central axis of the puncture needle for surgical suture insertion 20 or its extension line more reliably penetrates through the inside of the annular member 42. Further, it is preferable that the tip portion of the annular member 42 be V-shaped or U-shaped substantially around the tip point thereof, providing a surgical suture holding point 42a, where the distance across the loop is reduced. By providing such a surgical suture holding point 42a, the surgical suture S projected from the puncture needle for surgical suture insertion 20 can be more reliably held.

As the material for the rod-like member 41 of the stylet 40, a metal (such as stainless steel), a synthetic resin (for example, a polyolefin, such as polypropylene or polyethylene, or a fluorocarbon resin such as PTFE or ETFE) or the like can be preferably used. In addition, as the material for the annular member 42, elastic alloy wire, such as stainless steel wire (preferably, high tensile strength stainless steel wire for spring), piano wire (preferably, nickel-plated or chromium-plated piano wire), or super-elastic alloy wire, such as Ti—Ni alloy wire, Cu—Zn alloy wire, Cu—Zn—X alloy wire (X=Be, Si, Sn, Al, or Ga), or Ni—Al alloy wire can be preferably used.

On the proximal end side of the puncture needle for surgical suture gripping 30, a projection mechanism 70 for projecting the annular member 42 of the stylet 40, which is accommodated in the inside of the puncture needle for surgical suture gripping 30, from the distal end of the puncture needle for surgical suture gripping 30 is provided. The projection mechanism 70 includes a projection guide member 71 which is fixed to the fixing member 50 in a location where the proximal end of the puncture needle for surgical suture gripping 30 is open to the fixing member 50. This projection guide member 71 is cylindrically formed so as to movably guide a pushbutton 72, which is coaxially fixed at the proximal end of the stylet 40, in a linear direction (in a coaxial direction).

The basal end portion of the pushbutton 72, which provides a portion to be depressed by finger, is formed solid, however, the portion of the pushbutton 72 that follows the basal end portion thereof is formed in the shape of a hollow cylinder, being made of a synthetic resin which can be elastically deformed. The basal end portion of the rod-like member 41 is inserted into the hollow portion of the pushbutton 72 to be integrally fixed to the solid basal end portion thereof. In addition, in the inside of the projection guide member 71 between the end face of the distal end portion of the pushbutton 72 and the surface of the fixing member 50, a coil spring 73 is disposed with the rod-like member 41 being provided as the central axis. This coil spring 73 provides urging means for urging the pushbutton 72 in a direction toward the proximal end thereof along which the annular member 42 is accommodated in the inside of the puncture needle for surgical suture gripping 30.

In addition, on the circumference approximately in the middle of the pushbutton 72, a stopper 72a and a knob 72b, which are arranged vertically, being separated from each other, jut out. In order to accommodate these, the projection guide member 71 is provided with a locking groove 71a into which the stopper 72a is fitted, and a guide bore 71b in which the knob 72b is positioned on the upper end side thereof in a normal state in which the pushbutton 72 is not depressed toward the distal end side thereof. Thus, locking means is configured which, with the stopper 72a, knob 72b, locking groove 71a, and guide bore 71b, allows the pushbutton 72 to be temporarily seized in a position where it has been depressed toward the distal end side thereof.

As shown in FIG. 1 and FIG. 2, when the pushbutton 72 is depressed toward the distal end side thereof against the urging force of the coil spring 73, the portion of the pushbutton 72 on the distal end side thereof is elastically deformed toward the axis side, the stopper 72a being disengaged from the locking groove 71a, starting from the tapered surface side, and moved downward to be engaged with the upper end edge of the guide bore 71b. At this time, the knob 72b is moved toward the lower end side of the guide bore 71b. On the other hand, when the pushbutton 72 is to be returned to the original upward projected position, the knob 72b is depressed toward the inside of the projection guide member 71, which causes the stopper 72a to be disengaged from the upper end edge of the guide bore 71b, and to be moved upward by the urging force of the coil spring 73 to be fitted into the locking groove 71a.

As the material for the projection guide member 71, a metal (such as stainless steel), a synthetic resin (for example, a polyolefin, such as polypropylene or polyethylene, or a fluorocarbon resin such as PTFE or ETFE) or the like can be preferably used. In addition, as the material for the pushbutton 72, a synthetic resin (for example, vinyl chloride resin, or a polyolefin, such as polypropylene or polyethylene), or the like, which can be elastically deformed, is preferably used.

The fixing member 50 serves to fix the puncture needle for surgical suture insertion 20 and the puncture needle for surgical suture gripping 30 with the respective proximal ends thereof being open to the fixing member 50, whereby the puncture needle for surgical suture insertion 20 and the puncture needle for surgical suture gripping 30 are separated from each other a prescribed distance, and held in a state in which they are substantially parallel with each other. The distance between both provides a length over which the surgical suture S fixes the front abdominal wall and the splanchnic wall to each other, and is preferably, 10 mm to 30 mm or so, for example. If the length is in such a range, the front abdominal wall and the splanchnic wall can be sufficiently fixed to each other, and in addition, the resistance which would be encountered when the two puncture needles 20, 30 are punctured is not so high.

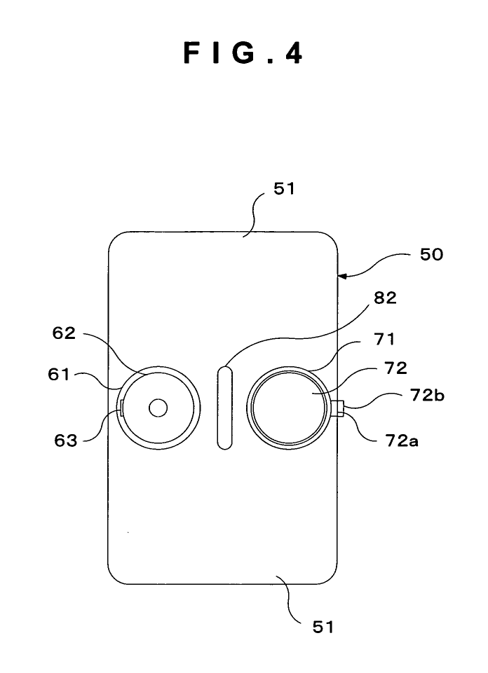

The fixing member 50 serves as a holding portion in puncturing the medical instrument 10, and as shown in FIG. 4, it is preferable that the fixing member 50 have a portion 51, 51 to which fingers can be applied. Further, as shown in FIG. 1 and FIG. 2, the fixing member 50 is preferably flat so as to make the holding thereof easy. The fixing member 50 is made of a synthetic resin, such as vinyl chloride resin, a polyolefin, such as polypropylene or polyethylene, or polycarbonate.

In addition, as shown in FIG. 1 and FIG. 2, it is preferable that the medical instrument 10 have a flat plate-like member 80 through which the puncture needle for surgical suture insertion 20 and the puncture needle for surgical suture gripping 30 are slidably penetrated. By providing such a flat plate-like member 80, it can be avoided that the distance between the puncture needle for surgical suture insertion 20 and the puncture needle for surgical suture gripping 30 is changed, and more specifically, that the distance between both is shortened at the time of puncture. In addition, because the flat plate-like member 80 is slidably formed, it will not obstruct the puncturing operation at the time of puncture.

As shown in FIG. 3, the flat plate-like member 80 may be made of a circular or polygonal plate material (not shown) as well as a rectangular plate material which bottom surface is flat, giving no stimulus, or the like, to the skin. Further, to the flat plate-like member 80, the distal ends of the operating lever 81 which extends substantially in parallel with the puncture needle for surgical suture insertion 20 and the puncture needle for surgical suture gripping 30 are fixed, and the operating lever 81 is slidably penetrated through the fixing member 50 on the way with a handle 82 being provided at the proximal end of the operating lever 81 for sliding operation of the flat plate-like member 80.

In the present embodiment, as shown in FIG. 3, the operating lever 81 is formed substantially in the shape of a letter U, by bending a metallic rod, and in the bent portion, the handle 82 is provided. In addition, the pair of distal end portions of the operating lever 81 is fixedly installed on the top surface side of the flat plate-like member 80 integrally therewith, and in the fixing member 50, a pair of small bores through which the pair of intermediate portions of the operating lever 81 is penetrated is provided.

Next, by means of the medical instrument 10 pertaining to the present embodiment, the operation of fixing the front abdominal wall and the splanchnic wall to each other, more specifically, the front abdominal wall and the gastric corpus front wall to each other will be described.

One operator inserts an endoscope into the stomach of a patient, and further supplies a sufficient quantity of air to fill the stomach with air for bringing the gastric corpus front wall into tight contact with the front abdominal wall. Then, another operator or an assistant disinfects the abdomen skin, confirms the position of the stomach by means of the transmitted light from the endoscope, and performs local anesthesia on the abdominal wall in this locus.

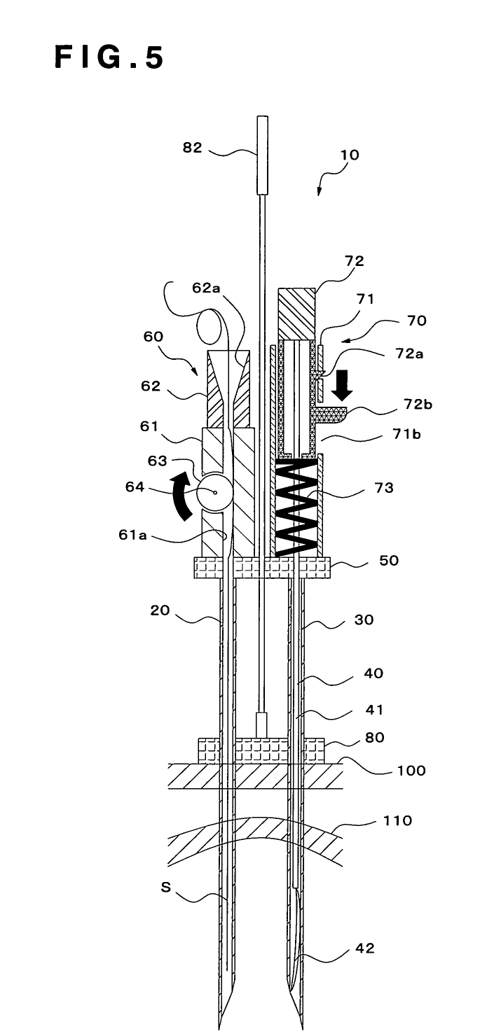

Then, as shown in FIG. 1, the medical instrument 10 is set up such that the annular member 42 of the stylet 40 is accommodated inside of the puncture needle for surgical suture gripping 30, and in addition, into the inside of the puncture needle for surgical suture insertion 20, the surgical suture S is inserted, but the end thereof is not projected from the distal end of the puncture needle for surgical suture insertion 20, and as shown in FIG. 5, the operator punctures this medical instrument 10 into the abdominal wall 100, and through the gastric corpus front wall 110, causes the puncture needle for surgical suture insertion 20 and the puncture needle for surgical suture gripping 30 to be projected into the stomach.

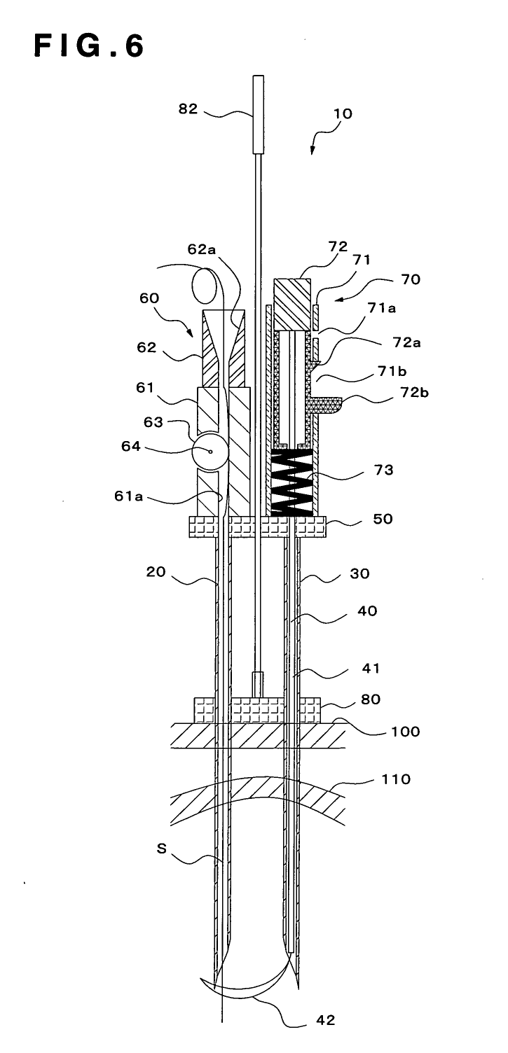

After the operator and the assistant confirming this state, as shown in FIG. 6, the operator uses the projection mechanism 70 to cause the annular member 42 provided at the distal end of the stylet 40 accommodated inside the puncture needle for surgical suture gripping 30 to be projected from the distal end of the puncture needle for surgical suture gripping 30. Then, the annular member 42 is extended toward the puncture needle for surgical suture insertion 20 such that the central axis of the puncture needle for surgical suture insertion 20 or its extension line penetrates through the inside of the annular member 42.

In operation of the projection mechanism 70, when, in FIG. 1, the pushbutton 72 is depressed toward the distal end side thereof against the urging force of the coil spring 73, the portion of the pushbutton 72 on the distal end side thereof is elastically deformed toward the axis side thereof, the stopper 72a being disengaged from the locking groove 71a, starting from the tapered surface side, and moved downward to be engaged with the upper end edge of the guide bore 71b as shown in FIG. 2. Thereby, the pushbutton 72 is temporarily held in the state in which it has been depressed toward the distal end side thereof, in other words, the annular member 42 has been projected from the distal end of the puncture needle for surgical suture gripping 30.

As shown in FIG. 3, when, as a result of depressing the pushbutton 72 in the projection mechanism 70 toward the distal end side thereof, the annular member 42 is projected from the distal end of the puncture needle for surgical suture gripping 30, the annular member 42 restores its natural state. In this state, the annular member 42 is maintained in the state in which it is extended toward the puncture needle for surgical suture insertion 20 such that the central axis of the puncture needle for surgical suture insertion 20 or its extension line penetrates through the inside of the annular member 42.

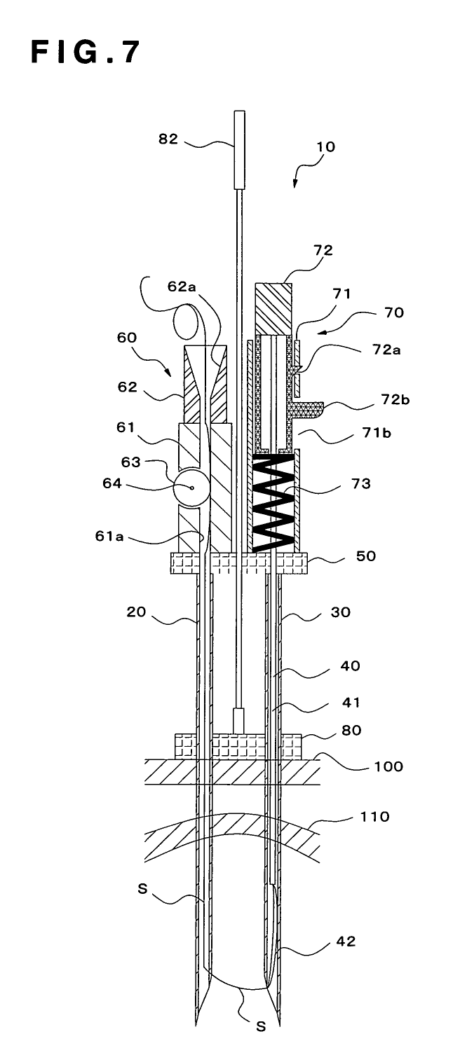

Then, as shown in FIG. 2, the feeding-out mechanism 60 is used to sequentially feed out the surgical suture S inserted into the inside from the proximal end of the puncture needle for surgical suture insertion 20 toward the distal end thereof to cause it to be projected from the distal end of the puncture needle for surgical suture insertion 20 and to be passed through the inside of the annular member 42. For operation of the feeding-out mechanism 60, the roller 63 can be appropriately turned by finger in a direction of arrow in FIG. 1. After the operator and the assistant confirming that the surgical suture S has been passed through the inside of the annular member 42, the operator operates the projection mechanism 70 to return the pushbutton 72 back to the original projected state as shown in FIG. 7.

In other words, in FIG. 2, by depressing the knob 72b toward the inside of the projection guide member 71, the stopper 72a is caused to be disengaged from the upper end edge of the guide bore 71b, and to be moved upward by the urging force of the coil spring 73 to be fitted into the locking groove 71a. With such an operation, the annular member 42 is accommodated into the inside of the puncture needle for surgical suture gripping 30, while, at this time, the annular space formed by the annular member 42 is gradually narrowed down with the shape being gradually changed into an ellipse.

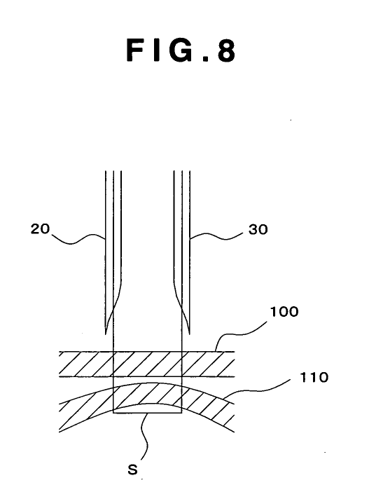

Thereby, the surgical suture S is held by the annular member 42 at the surgical suture holding point 42a thereof, and finally, as shown in FIG. 7, the portion of the surgical suture S that is held by the annular member 42 is accommodated inside the puncture needle for surgical suture gripping 30 together with the annular member 42. After the operator and the assistant confirming this state, the operator removes the medical instrument 10 from the patient. Then, as shown in FIG. 8, the distal end portion of the surgical suture S that has been inserted from the puncture needle for surgical suture insertion 20 is exposed to the outside of the human body.

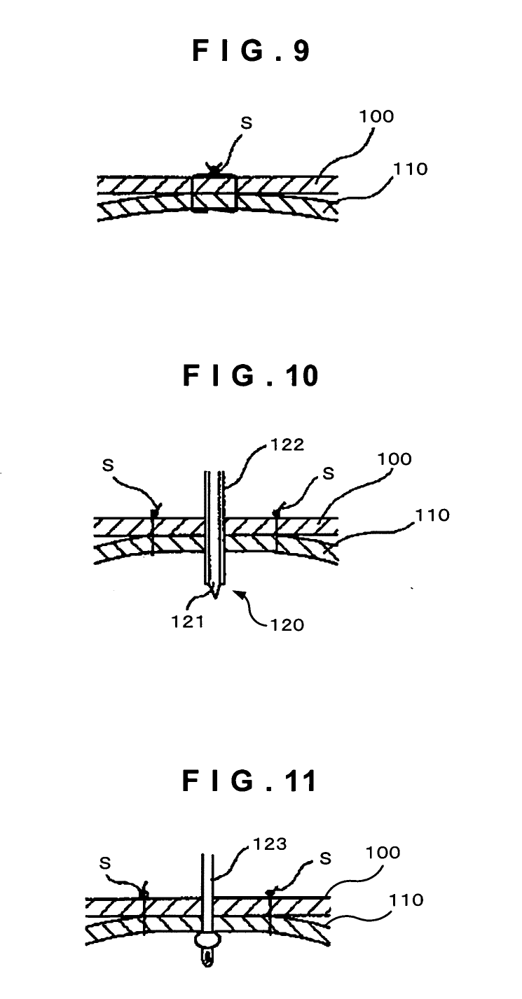

As shown in FIG. 9, the respective end portions of the exposed surgical suture S are ligated to each other. By this ligation, the gastric corpus front wall 110 and the abdominal wall 100 are fixed to each other. Further, in a location separated a prescribed distance, for example, 20 mm to 30 mm or so from this site of ligation with the surgical suture S, the medical instrument 10 is again punctured substantially in parallel, and as aforementioned, using the surgical suture S, the abdominal wall 100 and the gastric corpus front wall 110 are fixed to each other. Herein, the medical instrument 10 to be used for puncture may be the same as that which has been used for the aforementioned puncture, or that which is separately prepared.

Substantially in the middle of the two sites of ligation, as shown in FIG. 10, a puncture instrument 120 consisting of a metallic needle 121 having a blade at the tip and a plastic tube 122 into the inside of which this metallic needle 121 is inserted, and which is breakable along the axial direction is punctured, and after the metallic needle 121 being removed, a balloon catheter 123 is inserted into the inside of the plastic tube 122 as shown in FIG. 11. Then, after the operator and the assistant confirming that the distal end portion including the balloon portion of the balloon catheter 123 has reached the inside of the stomach, the balloon is inflated with distilled water, or the like, which is followed by pulling the basal end portion of the plastic tube 122 outward in two directions to break the plastic tube 122 along the axial direction before removing it from the patient.

Then, the balloon catheter 123 is pulled toward the basal end side thereof to bring the inflated balloon portion into tight contact with the gastric corpus front wall 110, and further an appropriate fixing instrument is applied to the portion of the balloon catheter 123 that is exposed to the skin surface for suturing it to fix the balloon catheter 123 to the gastric corpus front wall 110. By performing the above-described operations, the endoscopic gastrostomy is completed.

In the above-described endoscopic gastrostomy operation, the operation of feeding out the surgical suture S and the operation of projecting or withdrawing the annular member 42 from/into the distal end of the puncture needle for surgical suture gripping 30, which require fine and careful manipulation, can be reliably and easily performed by means of the feeding-out mechanism 60 and the projection mechanism 70, respectively, thus in case of making gastrostomy, even only one operator is capable of performing stomach wall fixing and endoscope manipulation, which allows only one operator or one operator and an assistant to efficiently carry out gastrostomy in safety. Especially, the projection mechanism 70 allows the annular member 42 to be easily projected to an exact position simply by depressing the pushbutton 72 by finger. In addition, the feeding-out mechanism 60 allows the surgical suture S to be easily fed out by an exact desired amount simply by turning the roller 63 by finger.

In addition, the medical instrument 10 pertaining to the present embodiment is provided with the flat plate-like member 80, thus it can be avoided that, at the time of puncture, the positional relationship between the puncture needle for surgical suture insertion 20 and the puncture needle for surgical suture gripping 30 is changed, and more specifically, that the distance between the respective puncture needles 20, 30 is changed (for example, shortened).

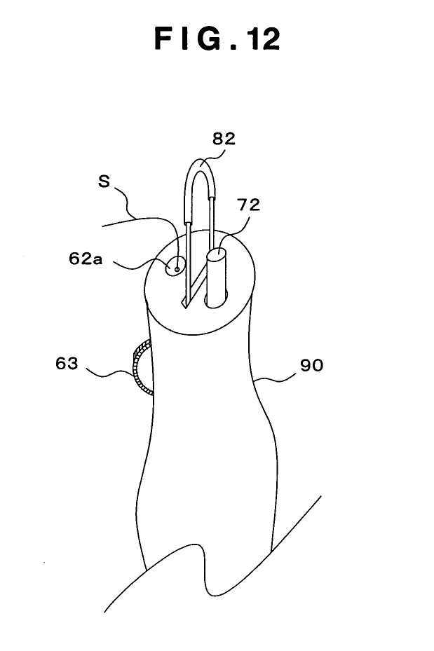

In addition, gripping the handle 82 to slide it will move the flat plate-like member 80 to a desired position, which can eliminate a possibility that, at the time of puncture, the flat plate-like member 80 may obstruct the puncture operation. Further, as shown in FIG. 12, by providing a cover body 90 which can accommodate the aforementioned components, such as the puncture needle for surgical suture insertion 20, the puncture needle for surgical suture gripping 30, and the fixing member 50, the medical instrument 10 can be gripped by one hand to easily carry out the operation.

Hereinabove, the embodiment of the present invention has been described with reference to the drawings, however, the specific configuration is not limited to the aforementioned embodiment, and any alteration and addition within the scope of the spirit of the present invention are possible to be incorporated in the present invention. For example, as an alternative embodiment, the medical instrument of the present invention may be configured to include two sets of the puncture needle for surgical suture insertion 20 and the feeding-out mechanism 60, and two sets of the puncture needle for surgical suture gripping 30 and the projection mechanism 70. The specific configuration of each set used may be the same as that in the aforementioned medical instrument 10.

More specifically, for example, the respective basal end portions of a first puncture needle for surgical suture insertion 20, a second puncture needle for surgical suture insertion 20, a first puncture needle for surgical suture gripping 30, and a second puncture needle for surgical suture gripping 30 can be fixed to the fixing member 50 such that they form the apexes of a tetragon. According to such a configuration, the abdominal wall 100 and the gastric corpus front wall 110 can be easily, rapidly, safely, and reliably fixed to each other in two places at the same time, thus the number of times of puncture invasiveness of this medical instrument 10 into the patient required in this fixing can be only one, which minimizes the burden on the patient.

In addition, it is preferable that a flat plate-like member 80 through which four puncture needles 20, 20, 30, 30 are slidably penetrated be provided. By providing such a flat plate-like member 80, it can be avoided that, at the time of puncture, the positional relationship among the four puncture needles is changed, and more specifically, that the distance between the respective puncture needles is changed (for example, shortened), and in addition, because the flat plate-like member 80 is slidable, it will not obstruct the puncturing operation at the time of puncture.

In addition, although the embodiment is configured such that the central axis of the puncture needle for surgical suture insertion 20 or its extension line penetrates through the inside of the annular member 42, the central axis of the puncture needle for surgical suture insertion 20, in other words, the distal end portion itself of the puncture needle for surgical suture insertion 20 may directly penetrate through the annular space which is formed by the annular member 42, provided that such a configuration will not obstruct the operation of projecting or accommodating the annular member 42 from/into the inside of the puncture needle for surgical suture gripping 30.

In addition, although the feeding-out mechanism 60 is configured such that, by turning a single roller 63, the surgical suture S is fed out, another configuration may be, for example, that in which two rollers which turnably engage with each other are provided, one roller being disposed to be butted against on the way of the surgical suture S, and the other roller being disposed to be capable of being turned from the outside, whereby, if the other roller is turned downward, the surgical suture S is fed out.

In addition, although the projection mechanism 70 is configured such that depression of the pushbutton 72 causes the annular member 42 to be projected, while the projected state is canceled by means of the knob 72b, which is provided independently of the pushbutton 72, another configuration may be, for example, that in which only depressing of the pushbutton 72 can alternately make projection and withdrawal operations, such as a well-known art with the knock-type mechanism for a general ball-point pen.

Furthermore, as the projection mechanism 70, instead of the configuration including the pushbutton 72, a configuration in which a roller which is turnably disposed, being butted against on the way of the stylet 40 to sequentially feed it out toward the distal end of the puncture needle for surgical suture gripping 30 through the turning operation thereof may be adopted.

### INDUSTRIAL APPLICABILITY

According to the medical instrument pertaining to the present invention, the operation of sequentially feeding out the surgical suture inserted into the inside of the puncture needle for surgical suture insertion toward the distal end thereof, and the operation of projecting the annular member of the stylet that is accommodated in the inside of the puncture needle for surgical suture gripping, from the distal end thereof, or withdrawing the projected annular member can be easily carried out even by one hand, whereby only one operator is capable of performing ligation and endoscope manipulation, which allows only one operator or one operator and an assistant to efficiently carry out operation in safety.

What is claimed is:

1. A medical instrument (10), comprising a puncture needle for surgical suture insertion (20); a puncture needle for surgical suture gripping (30) that is provided substantially in parallel with the puncture needle for surgical suture insertion (20), being separated therefrom a prescribed distance; a stylet (40) which is slidably inserted into the inside of the puncture needle for surgical suture gripping (30); and a fixing member (50) to which said puncture needle for surgical suture insertion (20) and said puncture needle for surgical suture gripping (30) are fixed, wherein
said stylet (40) is provided with an annular member (42) at the distal end thereof that is made of an elastic material, and is accommodatable in the inside of said puncture needle for surgical suture gripping (30), and when the annular member (42) is projected from the distal end of said puncture needle for surgical suture gripping (30), the annular member (42) is extended toward said puncture needle for surgical suture insertion (20) such that the central axis of said puncture needle for surgical suture insertion (20) or its extension line penetrates through the inside of the annular member (42);
on the proximal end side of said puncture needle for surgical suture insertion (20), a feeding-out mechanism (60) for sequentially feeding out a surgical suture (S) inserted into the inside of the puncture needle for surgical suture insertion (20) from the proximal end thereof toward the distal end thereof is provided; and
on the proximal end side of said puncture needle for surgical suture gripping (30), a projection mechanism (70) for projecting the annular member (42) of said stylet (40), which is accommodated in the inside of the puncture needle for surgical suture gripping (30), from the distal end of the puncture needle for surgical suture gripping (30) is provided.

2. The medical instrument (10) of claim 1, wherein said puncture needle for surgical suture insertion (20) is fixed to said fixing member (50) with the proximal end thereof being open to said fixing member (50);
said feeding-out mechanism (60) includes a feeding-out guide member (61) which is fixed to said fixing member (50) in a location where the proximal end of said puncture needle for surgical suture insertion (20) is open to said fixing member (50); and
said feeding-out guide member (61) is provided with a guide bore (61a) which is communicated with the proximal end of said puncture needle for surgical suture insertion (20), and a roller (63) which is turnably disposed, being butted against on the way of the surgical suture (S) that is guided into the inside of said puncture needle for surgical suture insertion (20) through the guide bore (61a), and which sequentially feeds out the surgical suture (S) toward the distal end of the puncture needle for surgical suture insertion (20) through the turning operation thereof.

3. The medical instrument (10) of claim 1, wherein said puncture needle for surgical suture gripping (30) is fixed to said fixing member (50) with the proximal end thereof being open to said fixing member (50);
said projection mechanism (70) includes a projection guide member (71) which is fixed to said fixing member (50) in a location where the proximal end of said puncture needle for surgical suture gripping (30) is open to said fixing member (50); and
said projection guide member (71) is cylindrically formed so as to movably guide a pushbutton (72) in a linear direction that is disposed on the proximal end side of said stylet (40), being provided with urging means (73) which urges said pushbutton (72) toward the proximal end thereof to accommodate said annular member (42) in the inside of said puncture needle for surgical suture gripping (30), and with locking means which allows said pushbutton (72), which is depressed toward the distal end thereof against the urging force of said urging means (73) to project said annular member (42) from the distal end of said puncture needle for surgical suture gripping (30), to be temporarily seized in a locking position.

4. The medical instrument (10) of claim 1, wherein said puncture needle for surgical suture insertion (20) and said puncture needle for surgical suture gripping (30) are provided with a flat plate-like member (80) through which they are slidably penetrated; and
to said flat plate-like member (80), the distal ends of an operating lever (81) which extends substantially in parallel with said puncture needle for surgical suture insertion (20) and said puncture needle for surgical suture gripping (30) are fixed, the operating lever (81) being slidably penetrated through said fixing member (50) on the way, and a handle (82) for sliding manipulation of said flat plate-like member (80) being provided at the proximal end of the operating lever (81).

5. The medical instrument (10) of claim 1, wherein two sets of said puncture needle for surgical suture insertion (20) and said feeding-out mechanism (60), and two sets of said puncture needle for surgical suture gripping (30) and said projection mechanism (70) are provided.
